(19) United States
(12) Patent Application Publication
(10) Pub. No.: US 2025/0087319 A1
(43) Pub. Date: Mar. 13, 2025

(54) INFORMATION INTERACTION METHOD, APPARATUS, DEVICE AND STORAGE MEDIUM

(71) Applicant: Beijing Zitiao Network Technology Co., Ltd., Beijing (CN)

(72) Inventors: Wei TU, Beijing (CN); Yihang Tang, Beijing (CN)

(21) Appl. No.: 18/882,658

(22) Filed: Sep. 11, 2024

(30) Foreign Application Priority Data
Sep. 11, 2023 (CN) 202311168004.6

(51) Int. Cl. G16H 10/60; G16H 15/00

(52) U.S. Cl. CPC G16H 10/60; G16H 15/00

(57) ABSTRACT

The embodiment of the disclosure provides an information interaction method, apparatus, device and a storage medium. The method includes: determining at least one question provided by a digital assistant to a user based on an information collection requirement of an electronic medical record; generating an electronic medical record of a consultation object based at least on interaction information between the user and the digital assistant, at least part of information in the electronic medical record being collected from the interaction information; and in accordance with a determination that a completeness of the electronic medical record satisfies a predetermined completeness condition, presenting the electronic medical record to medical personnel to determine a subsequent operation for the electronic medical record. In this way, a manner of more friendly acquisition of medical record information is provided for consultation object.

### CROSS REFERENCE

This application claims priority of the Chinese patent application No. 202311168004.6, entitled “INFORMATION INTERACTION METHOD, APPARATUS, DEVICE AND STORAGE MEDIUM” filed on Sep. 11, 2023, the entire content of which is incorporated herein by reference.

### FIELD

Example embodiments of the present disclosure generally relate to the field of computers, and in particular, to information interaction methods, apparatuses, devices, and computer-readable storage media.

### BACKGROUND

With the development of Internet technologies, it is also desirable in terms of health consultation to enable people to enjoy remote and online services. The method improves the supply of high-quality medical resources at high efficiency and low cost, helps the medical institution to expand the radiation range of the medical service, and enables the consultation object to obtain the medical guidance of the authoritative professional at any time. Currently, some websites, terminal applications, etc., attempt to provide online health consultation services. However, in online health consultation, an improvement in interactivity and expertise is desirable.

### SUMMARY

In a first aspect of the present disclosure, an information interaction method is provided. The method comprises: determining at least one question provided by a digital assistant to a user based on an information collection requirement of an electronic medical record; generating an electronic medical record of a consultation object based at least on interaction information between the user and the digital assistant, at least part of information in the electronic medical record being collected from the interaction information; and in accordance with a determination that a completeness of the electronic medical record satisfies a predetermined completeness condition, presenting the electronic medical record to medical personnel to determine a subsequent operation for the electronic medical record.

In a second aspect of the present disclosure, an apparatus for interacting information is provided. The apparatus comprises: a question determining module, configured to determine at least one question provided by a digital assistant to a user based on an information collection requirement of an electronic medical record; an electronic medical record generation module, configured to generate an electronic medical record of a consultation object based at least on interaction information between the user and the digital assistant, at least part of information in the electronic medical record being collected from the interaction information; and an electronic medical record presenting module, configured to, in accordance with a determination that a completeness of the electronic medical record satisfies a predetermined completeness condition, present the electronic medical record to medical personnel to determine a subsequent operation for the electronic medical record.

In a third aspect of the present disclosure, an electronic device is provided. The device includes at least one processing unit; and at least one memory coupled to the at least one processing unit and storing instructions for execution by the at least one processing unit. The instructions, when executed by the at least one processing unit, cause the device to perform the method of the first aspect.

In a fourth aspect of the present disclosure, a computer-readable storage medium is provided. The medium stores a computer program, and when the computer program is executed by the processor, the method in the first aspect is implemented.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2A illustrates a schematic diagram of an example of an interaction interface between a digital assistant and a user according to some embodiments of the present disclosure;

FIG. 2B and FIG. 2C are schematic diagrams illustrating an example of an interface for a user to confirm treatment information according to some embodiments of the present disclosure;

FIG. 3 illustrates a schematic diagram of an example of an interface for evaluating an electronic medical record according to some embodiments of the present disclosure;

FIG. 4A and FIG. 4B show schematic diagrams of an interface for editing an electronic medical record according to some embodiments of the present disclosure;

FIG. 5 illustrates a schematic diagram of an example of an interface for presenting an electronic medical record to a user according to some embodiments of the present disclosure;

FIG. 6 illustrates an example schematic diagram of an interface of a link corresponding to an entity word in an electronic medical record according to some embodiments of the present disclosure;

FIG. 7 illustrates an exemplary schematic diagram of an interface of a two-dimensional code of an electronic medical record according to some embodiments of the present disclosure;

FIG. 8 shows a flowchart of a process of information interaction according to some embodiments of the present disclosure;

FIG. 9 shows a block diagram of an apparatus of information interaction according to some embodiments of the present disclosure; and

FIG. 10 illustrates an electronic device in which one or more embodiments of the present disclosure may be implemented.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

In the description of the embodiments of the present disclosure, the terms “including” and the like should be understood as an open-end inclusion, i.e. “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below.

Herein, unless explicitly stated, “in response to A” performing one step does not imply that this step is performed immediately after “A”, but may include one or more intermediate steps.

It may be understood that the data involved in the technical solution (including but not limited to the data itself, the obtaining, using, storing or deleting of the data) should follow the requirements of the corresponding laws and regulations and related regulations.

It can be understood that before using the technical solutions disclosed in the embodiments of the present disclosure, relevant user should be informed of the types, use ranges, usage scenarios, and the like of the information related to the present disclosure in an appropriate manner according to relevant laws and regulations, and the authorization of the related user may be obtained, wherein the relevant users may include any type of rights body, such as individuals, businesses, and groups.

For example, when an active request of a user is received, prompt information is sent to the related user to explicitly prompt the related user, and the operation requested to be performed will need to obtain and use the information of the related user, so that the related user can autonomously select whether to provide information to software or hardware such as electronic device, application, server or storage medium and etc., executing the operation of the technical solution of the present disclosure according to the prompt information.

As an optional but non-limiting implementation, in response to receiving an active request of a related user, a manner of sending prompt information to the related user may be, for example, a pop-up window, and prompt information may be presented in a text manner in the pop-up window. In addition, the pop-up window may further carry a selection control for the user to select “agree” or “not agree” to provide information to the electronic device.

It may be understood that the foregoing notification and obtaining a user authorization process are merely illustrative, and do not constitute a limitation on implementations of the present disclosure, and other manners of meeting related laws and regulations may also be applied to implementations of the present disclosure.

As used herein, the term “model” may learn an association relationship between respective inputs and outputs from training data such that a corresponding output may be generated for a given input after training is complete. The generation of the model may be based on machine learning techniques. Deep learning is a machine learning algorithm that processes inputs and provides corresponding outputs by using a multi-layer processing unit. The neural network model is one example of a deep learning based model. As used herein, a “model” may also be referred to as a “machine learning model,” a “learning model,” a “machine learning network,” or a “learning network,” which terms are used interchangeably herein.

In an online health consultation process, information of a consultation object needs to be collected and a medical record report is generated. In the current solution, an electronic form is provided for a user (for example, a consultation object himself or herself), and the user may fill in the electronic form with the disease information of the consultation object. Further, the medical record is generated based on the information that the user fills in the electronic form. However, because the user lacks medical professional knowledge, it is often impossible to accurately and comprehensively describe the condition of the consultation object, so the information filled in the electronic form may not be comprehensive and accurate enough. Resulting in that the doctor cannot obtain complete medical record information.

According to an embodiment of the present disclosure, an information interaction solution is provided. In this solution, at least one question provided by the digital assistant to the user is determined based on the information collection requirement of the electronic medical record. Then, based on the interaction information between the user and the digital assistant, an electronic medical record of the consultation object is generated, and at least part of the information in the generated electronic medical record is collected from the interaction information. If the completeness of the electronic medical record satisfies the predetermined completeness condition, the electronic medical record is presented to medical personnel, and the medical personnel may perform subsequent operations for the electronic medical record. In this way, a way of more friendly collection of medical record information is provided for the consultation object. In addition, for the medical personnel, the electronic medical record can be automatically generated more quickly, and the working efficiency of medical personnel can be improved.

Some example embodiments of the present disclosure will be described in detail below with reference to the accompanying drawings.

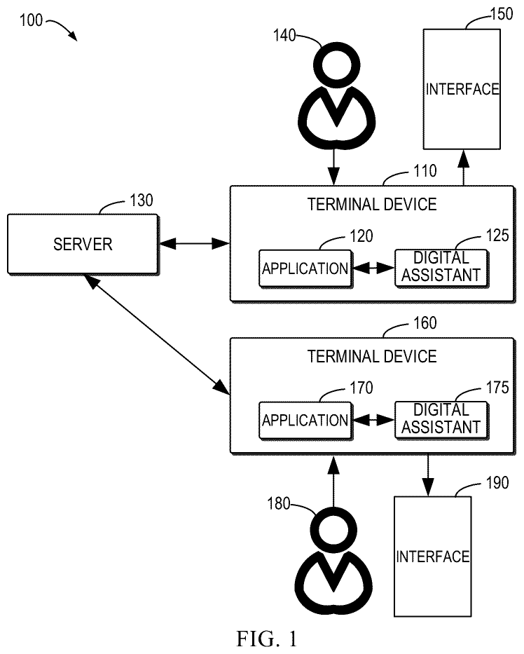

FIG. 1 illustrates a schematic diagram of an example environment 100 in which embodiments of the present disclosure can be implemented. In this example environment 100, a terminal device 110 on a side of a user (for example, a consultation object) and a terminal device 160 on a medical personnel side are included. An application 120 is installed in the terminal device 110. The user 140 may interact with the application 120 via the terminal device 110 and/or an attachment device of the terminal device 110. The user 140 is a health consultation object. For example, the user 140 may be a consultation object himself or herself, or may be other users such as a friend or a relative of the consultation object. Similar to the user side, an application 170 is installed in the terminal device 160 on the medical personnel side. The medical personnel 180 may interaction with the application 170 via the terminal device 160 and/or an attachment device of the terminal device 160.

In some embodiments, the application 120 may be downloaded, installed in the terminal device 110. In some embodiments, the application 120 may also be accessed in other manners, such as web page access. In the environment 100 of FIG. 1, if the application 120 is launched, the terminal device 110 may present the interface 150 of the application 120. The interface 150 may include various user interfaces that may be provided by the application 120. The interface 150 is also sometimes referred to as a client interface.

Similarly, application 170 may be downloaded, installed on the terminal device 160. In some embodiments, the application 170 may also be accessed in other manners, such as web page access. In the environment 100 of FIG. 1, if the application 170 is launched, the terminal device 160 may present the interface 190 of the application 170. The interface 190 may include various user interfaces that may be provided by the application 170. Interface 190 is also sometimes referred to as a client interface.

In embodiments of the present disclosure, application 120 includes any suitable application capable of providing a health consultation service. In some embodiments, the application 120 may implement a conversation interaction with the user 140 based on the digital assistant 125 to provide a health consultation service to the user 140. In some embodiments, the application 120 may provide other services in addition to the interaction of the digital assistant 125, which is not limited in this respect.

Similarly, any suitable application capable of providing a health consultation service is included in the application 170. In some embodiments, the application 170 may implement a conversation interaction with the medical personnel 180 based on the digital assistant 175 to provide a health consultation service to the user 140. In some embodiments, the application 170 may provide other services in addition to the interaction of the digital assistant 175, which is not limited in this respect.

In some embodiments, terminal device 110 may communicate with server 130 to enable provision of services to applications 120 and/or digital assistant 125. Similarly, terminal device 160 may also communicate with server 130 to enable provision of services to applications 170 and/or digital assistant 175. The terminal device 110 and the terminal device 160 may be any type of mobile terminal, fixed terminal, or portable terminal, including a mobile phone, a desktop computer, a laptop computer, a notebook computer, a netbook computer, a tablet computer, a media computer, a multimedia tablet, a personal communication system (PCS) device, a personal navigation device, a personal digital assistant (PDA), an audio/video player, a digital camera/camcorder, a television receiver, a radio broadcast receiver, an electronic book device, a gaming device, or any combination of the foregoing, including accessories and peripherals of these devices, or any combination thereof. In some embodiments, the terminal device 110 and the terminal device 160 can also support any type of interface for a user (such as a “wearable” circuit, etc.). The server 130 may be various types of computing systems/servers capable of providing computing power, including, but not limited to, mainframes, edge computing nodes, computing devices in a cloud environment, and the like.

It should be understood that the structures and functions of the various elements in the environment 100 are described for exemplary purposes only and do not imply any limitation to the scope of the present disclosure.

In embodiments of the present disclosure, the digital assistant 125 and the digital assistant 175 are used to interact with a user. An interaction interface, also referred to as an interaction window or a conversation window, between the user and the digital assistant 125 and the digital assistant 175 is presented in the client interface. The digital assistant 125 and the digital assistant 175 serve as a smart assistant, and have an intelligent conversation and information processing capability. In the interaction interface, the user inputs an interaction message, and the digital assistants 125 and 175 provide a reply message in response to the user input. In applications 120 and 160, the digital assistants 125 and 175 may be activated or awakened in an appropriate manner (e.g., shortcut keys, buttons, or voice) to present an interaction interface with the user. The interaction interface may include an interface element for information interaction, such as an input box, a message list, a message bubble, and the like.

In embodiments of the present disclosure, digital assistant 125 is used to provide services related to health consultation, which may sometimes be referred to as a “digital doctor”. The user may send a health consultation request to the digital assistant 125 by using a simple text or voice input, and may obtain a real-time reply of the digital assistant 125 for medical knowledge science questioning and answering, health consultation, or general health management. In this way, the digital assistant 125 can be used to achieve high efficiency and low cost to improve the supply of high-quality medical resources, help the medical institution to expand the radiation range of the medical service, and enable the consultation object to obtain the guidance of the authoritative professional at any time.

In some embodiments, the digital assistant 125 may determine a reply to the user's question by means of a model. In some embodiments, the model may be trained based on a general corpus and a specialized corpus for the medical field, enabling reachable, credible, understandable medical knowledge publicity and health consultation guidance. The dedicated corpus in the medical field may include, for example, national and international authoritative clinical guidelines, documents, textbooks, clinical doctor-patient communication question-answer data, and the like. In some embodiments, the training of the medical safety related mechanism can be strengthened during model training, so that the model can have the capability of providing credible health consultation suggestions, and a comprehensive safety test is passed. In some embodiments, when the performance of the digital assistant 125 is evaluated, the capability of the digital assistant 125 in terms of communication expression ability and humanistic care and friendliness may also be evaluated, so that the digital assistant 125 can implement aspects such as empathy with a consultation object, understanding requirements of consultation object, detailed and patient communication, and the like, which is conducive to alleviating the problems of short communication time and insufficient communication.

The model may be deployed on the terminal device 110 or may be accessed by the terminal device 110. Alternatively, the terminal device 110 may communicate with the model to provide the application management service for the user 140 using the model. For example, the terminal device 110 may send a prompt input to the model based on the interaction information between the user 140 and the digital assistant 125, and obtain a processing result of the model. Alternatively or additionally, the digital assistant 125 itself may also be obtained based on a model. The digital assistant 125 may process the session message input by the user 140 to obtain a requirement of the user 140, for example, a problem expected to be consulted by the user 140, and the like. The model may be configured locally or independently of terminal device terminal device 110 according to requirements, and the present disclosure is not intended to be limited thereto.

Some example embodiments of the present disclosure will be described below with continued reference to the accompanying drawings.

In some embodiments of the present disclosure, the digital assistant 125 may guide the user 140 to perform questioning and answering in a natural conversation to obtain medical record information. For this, it is necessary to determine the questions that the digital assistant 125 needs to ask to the user 140 in the course of a questioning and answering. In an implementation of the present disclosure, at least one question provided by the digital assistant 125 to the user 140 may be determined based on the information collection requirement of the electronic medical record, and these questions are presented to the user 140. In some embodiments, the digital assistant 125 may generate a reply for the user 140 based on the model. In this way, the information used to generate the electronic medical record is collect in the manner of guiding the user 140 to answer the question.

In the embodiments of the present disclosure, the information collection requirement of the electronic medical record may be set as needed. The content of the electronic medical record that needed to be filled in are different for each hospital, the settings of these questions may be different for digital assistants applied to different hospitals.

For example, the information collection requirement of the electronic medical record may include basic information of the consultation object, some disease condition of the consultation object, an allergy history of the consultation object, and the like. The questions to be asked may be generated based on these requirements. The question to be asked by the digital assistant may be, for example, “please describe your problem in detail?”, “is there any other uncomfortable symptom?”.

The electronic medical record of the consultation object may then be generated based at least on the interaction information between the user 140 and the digital assistant 125. At least part of the generated electronic medical records is collected from the interaction information.

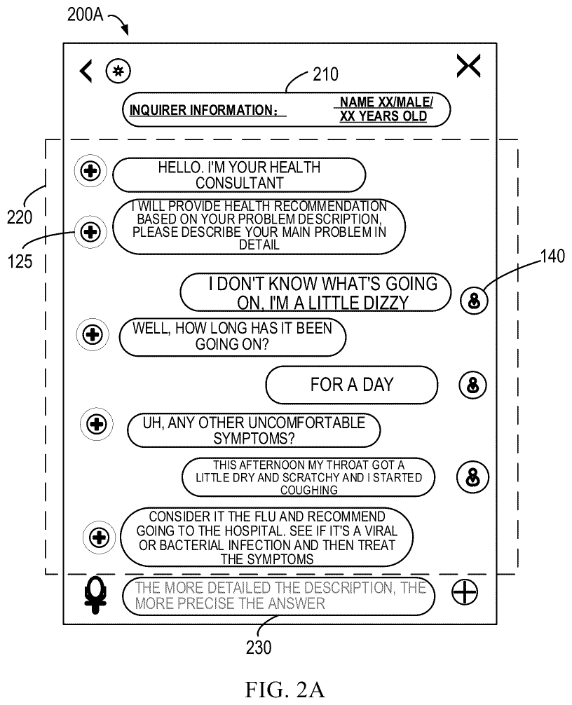

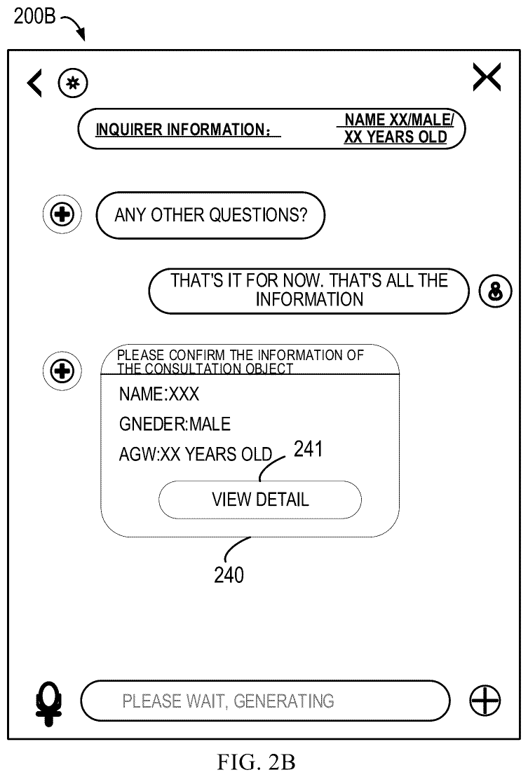

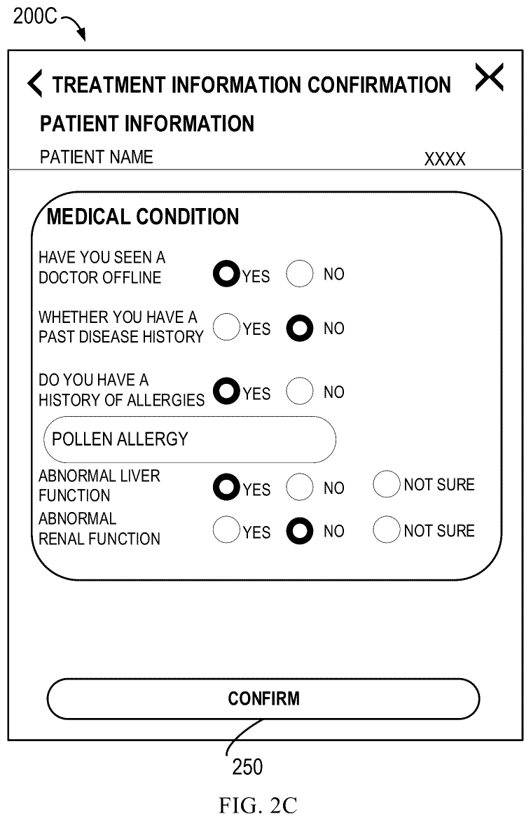

For example, referring to FIG. 2A, an interaction interface 200A is presented between the digital assistant 125 and the user 140. The interaction information 230 is generated between the digital assistant 125 and the user 140 through a multi-round interaction. The interaction interface 200A further includes basic information 210 of the user 140, for example, a name, a gender, and an age of the user. It may be understood that the basic information 210 may be obtained by a user filling in a form, or may be obtained by interaction between the digital assistant 125 and the user 140. In addition, in the interaction interface 200A of FIG. 2A, the user 140 may further input the interaction information through the input box 230.

Optionally, before generating the electronic medical record, the digital assistant 125 may first present the medical record information obtained from the interaction information 230 to the user 140, and the user 140 performs confirmation on the information.

For example, referring to FIGS. 2B and 2C. Digital assistant 125 may present interaction interface 200B. In this interaction interface 200B, a prompt box 240 is presented. The prompt box 240 shows the treatment information that needs to be confirmed by the user 140. The prompt box 240 also presents a “view detail” control 241, and the user 140 may click on the control 241 to further view the treatment information that needs to be confirmed. If the user performs a triggering operation for the control 241, the interaction interface 200C shown in FIG. 2C may be presented. The interaction interface 200C shows the patient's information and the medical condition information. This information may be obtained based on an interaction between the user 140 and the digital assistant 125. The “confirm” control 250 is presented in the interaction interface 200C. If the user triggers the control 250, for example, clicks the control 250, it indicates that the user the user confirms the treatment information. Thereafter, the electronic medical record may be further generated based on the treatment information confirmed by the user.

Next, if the completeness of the electronic medical record satisfies the predetermined completeness condition, the electronic medical record is presented to medical personnel. The medical personnel may perform subsequent operations on the generated medical records.

The complete electronic medical record generally needs to include medical condition information related to the consultation object and medical diagnosis related information. In some embodiments, if it is determined that the completeness of the medical condition information in the electronic medical record satisfies the predetermined completeness condition, then the electronic medical record may be presented to medical personnel.

That is, the interaction information between the digital assistant 125 and the user 140 includes substantially complete information, for example, a basic condition of the consultation object. In this case, the part of information that needs to be collected in the electronic medical record, for example, the basic condition of the consultation object, may be obtained from the interaction information (for example, the interaction information 230 in FIG. 2A), and then the electronic medical record may be presented to the medical personnel 180. In some embodiments, medical diagnostic information that needs to be included in the electronic medical record, such as diagnosis, treatment recommendations, prescriptions, etc., may be filled in by medical personnel 180.

In some embodiments, more complete and comprehensive medical record information can be obtained based on the interaction information between the digital assistant 125 and the user 140. In this case, the electronic medical record may be directly generated based on the interaction information between the digital assistant 125 and the user 140, and presented to the medical personnel 180. The medical staff 180 only needs to check the medical record information in the generated electronic medical record and confirm whether the medical record information passes the check. In this scenario, the medical personnel 180 only needs to perform the confirmation operation on the electronic medical record, so that the operation burden of the medical personnel 180 can be greatly relieved, and the working efficiency of the medical personnel 180 can be improved.

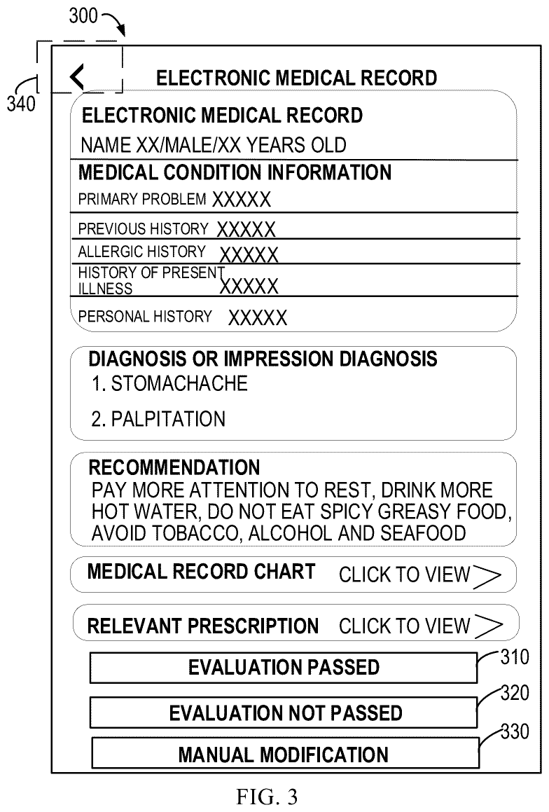

As an example, FIG. 3 is an evaluation interface 300 of an electronic medical record provided by the terminal device 160. The interface 300 presents detailed information of the electronic medical record and provides an evaluation passed control 310, an evaluation not passed control 320, and a manual modification control 330. In some embodiments, if the medical personnel 180 clicks the evaluation passed control 310, it indicates that the medical personnel 180 confirms that the detail information of the electronic medical record displayed by the interface 300 is confirmed to be correct, an electronic medical record including the displayed detail information may be generated.

In some embodiments, the confirmed electronic medical record may also be stored in association with the consultation object or the user in response to receiving a confirmation on the electronic medical record by medical personnel 180 (e.g., the triggering operation of the evaluation passed control 310 in FIG. 3). As mentioned above, the user 140 performing the health consultation and the consultation object may not be the same user. Thus, confirmed electronic medical records may be stored in association with the consultation object and/or the users 140 performing the health consultation. In this way, it is convenient to reserve electronic medical records associated with different consultation objects and/or user, so as to facilitate the viewing of the consultation object and/or user.

In some embodiments, the confirmed electronic medical record may also be provided in an interaction interface between the user 140 with the digital assistant 125 for viewing by the user 140.

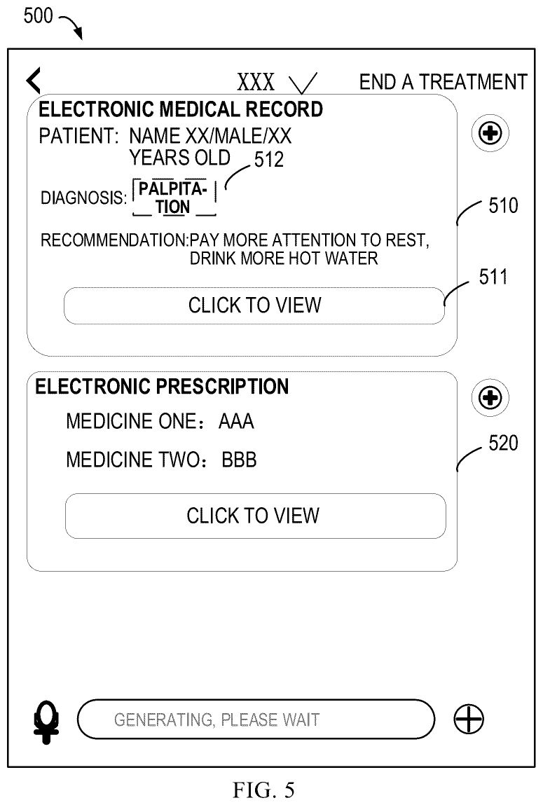

As an example, referring to FIG. 5, the electronic medical record 510 is displayed in the interaction interface 500 between the user 140 and the digital assistant. The user 140 may click “click to view” control 511 to view the details information of the electronic medical record 510. Optionally, the interface 500 further displays an electronic prescription 520 for this health consultation.

In some embodiments, after the generated electronic medical record is presented to the medical personnel 180, the medical personnel 180 may determine whether a further interaction between the digital assistant 125 and the user 140 is required according to needs, in order to obtain more interaction information, thereby modifying the electronic medical record. If more interaction information is to be obtained, further interaction between the digital assistant and the user may be initiated in response to receiving a return of the electronic medical record by medical personnel 180. The modified electronic medical record may then be generated based on further interaction information between the user with the digital assistant.

Still referring to FIG. 3. For example, if medical personnel 180 clicks on an evaluation not passed control 320, the click operation may trigger further interaction by digital assistant 125 with the user 140. For another example, if the medical personnel 180 clicks on the return control 340, the operation may also trigger to initiate further interaction between the digital assistant 125 and the user 140.

In some embodiments, the medical personnel 180 may also edit the electronic medical record directly. Specifically, the medical personnel may trigger an editing operation for the electronic medical record, and further provide a medical record editing interface to receive the editing content for the electronic medical record in response to receiving the editing operation on the electronic medical record by the medical personnel.

For example, referring still to FIG. 3. The editing operation triggered by the medical personnel 180 for the electronic medical record may be, for example, the medical personnel 180 clicking the “manual modification” control 330. In response to this, the medical record editing interface may be provided, and the medical personnel 180 may modify, delete, add, etc. the content of the electronic medical record. For example, for the scenario where medical diagnosis information is not included in the electronic medical record mentioned above, the medical personnel 180 may provide medical diagnosis information in the medical record editing interface. Further, the medical personnel 180 may also provide an electronic prescription in the medical record editing interface (the electronic prescription may be, for example, shown as the aforementioned prescription 520).

In some embodiments, the medical record editing interface includes a viewing entrance for a plurality of interaction messages between the user and the digital assistant. The viewing entrance may be, for example, a predetermined control. The editing operation for the electronic medical record triggered by the medical personnel 180 may include a trigger operation for the viewing entrance. The terminal device 160 may present a plurality of interaction messages in response to a trigger operation for the viewing entrance. Also, the plurality of interaction messages presented may be selected by medical personnel. The medical personnel may select one or more interaction information, and the selected at least one interaction message may be used to update the electronic medical record.

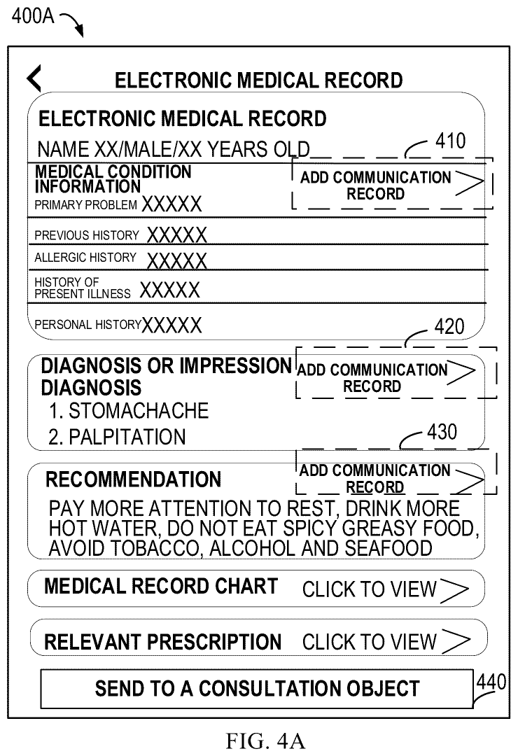

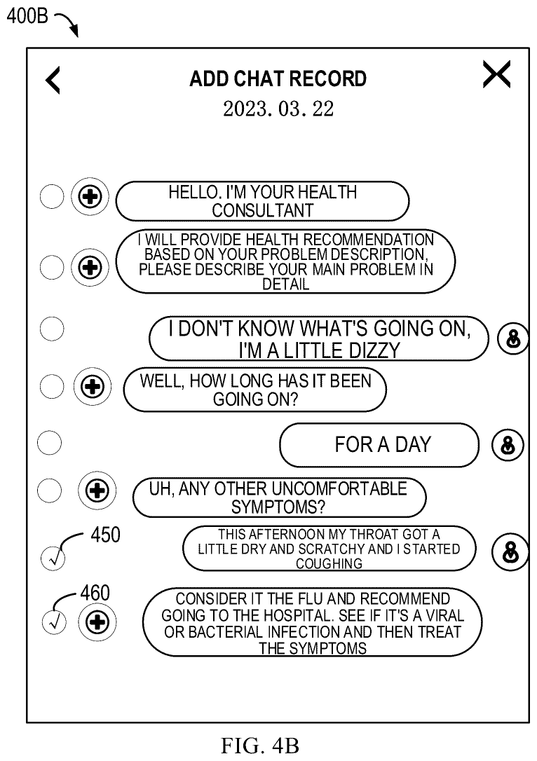

As an example, FIG. 4A provides an example of a medical record editing interface. In the example of FIG. 4A, detailed information of the electronic medical record is displayed in the medical record editing interface 400A. The electronic medical record specifically includes parts such as “medical condition information”, “diagnosis or impression diagnosis”, and “treatment suggestion”. And, for these parts, three control of “add communication record” 410, 420 and 430 are provided, which can trigger the viewing of a plurality of messages between the user 140 and the digital assistant 125. In other words, the three controls are viewing entrances of the interaction messages between the user 140 and the digital assistant 125. The operation of the three controls 430 may trigger a presentation of a plurality of interaction information. For example, FIG. 4B is an interaction message interface after any one of controls 410, 420, and 430 is triggered. The interface 400B presents a plurality of interaction messages between the user 140 and the digital assistant 125. The medical personnel 180 may select an interaction message from the plurality of interaction messages to update the electronic medical record. In the example of FIG. 4B, medical personnel 180 selects two interaction messages 450 and 460. It will be appreciated that the operation of controls 410, 420, and 430 may trigger the viewing of the same or different interaction messages. For example, the triggering operation on any one control of the three controls may view all interaction messages between the user 140 and the digital assistant 125. As another example, an operation of one of the controls only triggers an associated portion of the interaction messages. Specifically, the operation of the control 410 can trigger the display of an interaction message related only to the medical condition information. Operation of the control 420 can trigger a display of only diagnostic related interaction messages. The operation of the control 430 can trigger a display of an interaction message related only to the treatment recommendation.

In some embodiments, after the medical personnel 180 completes editing the electronic medical record, the medical personnel 180 may further send the edited electronic medical record to the user 140. For example, the interface shown in FIG. 4 displays a “send to a consultation object” control 440. The user clicking the control 440 may trigger displaying the edited electronic medical record to the user 140. On the user 140 side, the terminal device 110 may provide the interface 500 shown in FIG. 5 as previously mentioned.

Electronic medical records often contain professional vocabularies, which are not easily understood for common user. In view of this, in an electronic medical record, at least one entity word is labeled with a corresponding entry link, and each entry link is used to access an entry page corresponding to the entity word. The entity word in the embodiments of the present disclosure may be some medical terms or the like. In this way, the entity word is marked in the medical record report, and the entity word is bound to the corresponding knowledge content. The user can conveniently click and view the explanation of related entity words, so that the consultation object can intuitively understand the medical record report.

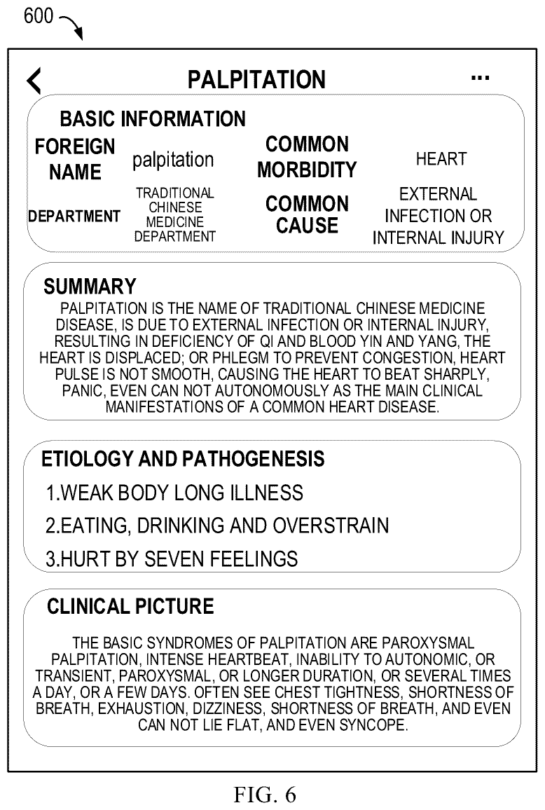

In some implementations of this embodiment, the entity word may be highlighted in a manner different from other words. For example, entity words are bolded, displayed with colors different from other words, etc. As an example, referring still to FIG. 5, the electronic medical record shown in the user 140 side terminal device 160 includes the entity word 512 “palpitation”. If the user 140 clicks on the entity word, the knowledge of the entity word may be displayed. Exemplarily, referring to FIG. 6, detailed information of the entity word 512 “palpitation” may be displayed as the interface 600.

In the embodiments of the present disclosure, all the entity words may be obtained by enumerating through matching the keyword included in the medical record information with a predetermined word bank (the predetermined word bank may be a medical/health word bank). Optionally, an embodiment of the present disclosure further provides an existing mechanism of an entity word, where the exiting mechanism herein means that some entity words are converted into non-entity words when a certain condition is met. As an implementation, for a certain entity word, a number of times that a plurality of users click on the entity word to view a knowledge link corresponding to the entity word may be counted, and if the number of clicks is relatively large. The entity word may then continue to be presented to the user. If the frequency of clicking the entity word by the user is relatively small, the entity word is converted into a non-entity word and displayed to the user. It should be noted that the user participating in statistics may be all user that generate the electronic medical record. As a finer granularity implementation, user participating in statistics may also be user with the same user portray. This is not limited in the embodiments of the present disclosure.

In the embodiments of the present disclosure, for a scenario in which a certain entity word determined in an electronic medical record appears repeatedly for a plurality of times, the word may be presented as an entity word for one time, and other scenario may be presented as non-entity words.

In the embodiment of the present disclosure, if a plurality of entity words are densely determined in the same piece of content in the electronic medical record, the words which are clicked relatively frequent may be obtained based on the statistics may be presented as entity words, and the other words are presented as non-entity words.

In some embodiments, at least one of the following: an access link of the electronic medical record, or an access link of the interaction information is provided in a page associated with the electronic medical record. The access link of the electronic medical record and/or the interaction information may be presented directly in the form of a webpage address or in the form of a two-dimensional code.

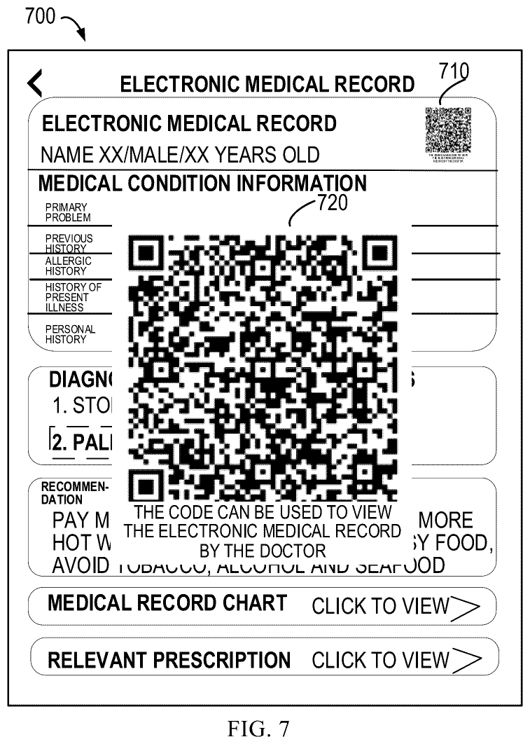

As an example, referring to FIG. 7, the two-dimensional code 710 of the electronic medical record is presented in the page 700 associated with the electronic medical record. The user 140 clicks on the two-dimensional code 710, the two-dimensional code is enlarged and displayed as a two-dimensional code 720, and the two-dimensional code 720 is available for scanning to view the electronic medical record or the interaction information.

In this embodiment of the present disclosure, the generated access link may be quickly accessed by the user 140 at the next treatment. For example, viewed by an offline doctor when performing treatment offline or viewed by another doctor when performing treatment online. Therefore, the medical record report can be conveniently and quickly checked at next treatment, and the interaction efficiency of the medical record information is greatly improved.

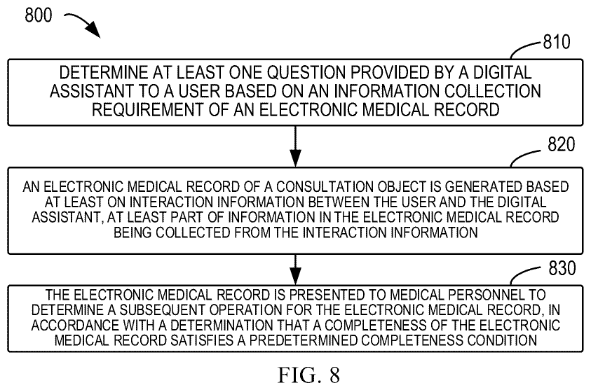

FIG. 8 shows a flowchart of a process 800 of information interaction according to some embodiments of the present disclosure. The process 800 may be implemented, for example, at server 130, or partially or entirely at terminal device 110 or 160. The process 800 is described below with reference to FIG. 1.

At block 810, at least one question provided by a digital assistant to a user is determined based on an information collection requirement of an electronic medical record.

At block 820, an electronic medical record of a consultation object is generated based at least on interaction information between the user and the digital assistant, at least part of information in the electronic medical record being collected from the interaction information.

At block 830, in accordance with a determination that a completeness of the electronic medical record satisfies a predetermined completeness condition, the electronic medical record is presented to medical personnel to determine a subsequent operation for the electronic medical record.

In some embodiments, the electronic medical record at least comprises medical condition information related to the consultation object, and wherein presenting the electronic medical record to medical personnel comprises: in accordance with a determination that completeness of the medical condition information in the electronic medical record satisfies a predetermined completeness condition, presenting the electronic medical record to medical personnel.

In some embodiments, the process 800 further comprises: in response to receiving a confirmation on the electronic medical record by the medical personnel, storing the confirmed electronic medical record in association with the consultation object or the user.

In some embodiments, the process 800 further comprises: providing the confirmed electronic medical record in an interaction interface between the user and the digital assistant for viewing by the user.

In some embodiments, the process 800 further comprises: in response to receiving a return of the electronic medical record by the medical personnel, initiating a further interaction between the digital assistant and the user; and generating a modified electronic medical record based on information of the further interaction between the user and the digital assistant.

In some embodiments, the process 800 further comprises: in response to receiving an editing operation on the electronic medical record by the medical personnel, providing a medical record editing interface to receive editing content for the electronic medical record.

In some embodiments, the medical record editing interface comprises a viewing entrance for a plurality of interaction messages between the user and the digital assistant. The process 800 further comprises: presenting the plurality of interaction messages based on a triggering operation on the viewing entrance, the plurality of interaction messages being selectable; receiving a selection on at least one interaction message in the plurality of interaction messages by the medical personnel; and updating the electronic medical record with the selected at least one interaction message.

In some embodiments, in the electronic medical record, at least one entity word is annotated with a respective entry link, and each entry link is used for accessing an entry page corresponding to an entity word.

In some embodiments, at least one of the following: an access link of the electronic medical record, or an access link of the interaction information is provided in a page associated with the electronic medical record.

In some embodiments, the digital assistant generates a reply for the user based on a model.

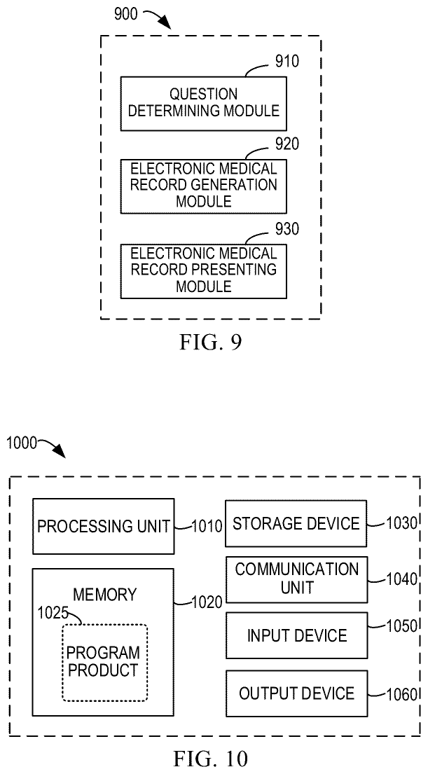

FIG. 9 is a schematic structural block diagram of an apparatus 900 for interacting information according to some embodiments of the present disclosure. The apparatus 900 may be implemented as or included in the server 130, the terminal device 110, or the terminal device 160 of FIG. 1. The various modules/components in the apparatus 900 may be implemented by hardware, software, firmware, or any combination thereof.

As shown in FIG. 9, the apparatus 900 includes a question determining module 910, configured to determine at least one question provided by a digital assistant to a user based on an information collection requirement of an electronic medical record. The apparatus 900 further includes an electronic medical record generation module 920, configured to generate an electronic medical record of a consultation object based at least on interaction information between the user and the digital assistant, at least part of information in the electronic medical record being collected from the interaction information. The apparatus 900 further includes an electronic medical record presenting module 930, configured to, in accordance with a determination that a completeness of the electronic medical record satisfies a predetermined completeness condition, present the electronic medical record to medical personnel to determine a subsequent operation for the electronic medical record.

In some embodiments, the electronic medical record at least comprises medical condition information related to the consultation object, and wherein presenting the electronic medical record to medical personnel comprises: in accordance with a determination that completeness of the medical condition information in the electronic medical record satisfies a predetermined completeness condition, presenting the electronic medical record to medical personnel.

In some embodiments, the apparatus 900 further includes a storage module configured to, in response to receiving a confirmation on the electronic medical record by the medical personnel, store the confirmed electronic medical record in association with the consultation object or the user.

In some embodiments, the apparatus 900 further includes an electronic medical record providing module configured to provide the confirmed electronic medical record in an interaction interface between the user and the digital assistant for viewing by the user.

In some embodiments, the apparatus 900 further includes a medical record modification module configured to, in response to receiving a return of the electronic medical record by the medical personnel, initiate a further interaction between the digital assistant and the user; and generate a modified electronic medical record based on information of the further interaction between the user and the digital assistant.

In some embodiments, the apparatus 900 further includes a medical record editing module configured to, in response to receiving an editing operation on the electronic medical record by the medical personnel, provide a medical record editing interface to receive editing content for the electronic medical record.

In some embodiments, the medical record editing interface comprises a viewing entrance for a plurality of interaction messages between the user and the digital assistant. The medical record editing module is further configured to present the plurality of interaction messages based on a triggering operation on the viewing entrance, the plurality of interaction messages being selectable; receive a selection on at least one interaction message in the plurality of interaction messages by the medical personnel; and update the electronic medical record with the selected at least one interaction message.

In some embodiments, in the electronic medical record, at least one entity word is annotated with a respective entry link, and each entry link is used for accessing an entry page corresponding to an entity word.

In some embodiments, at least one of the following: an access link of the electronic medical record, or an access link of the interaction information is provided in a page associated with the electronic medical record.

In some embodiments, the digital assistant generates a reply for the user based on a model.

FIG. 10 illustrates a block diagram of an electronic device 1000 in which one or more embodiments of the present disclosure may be implemented. It should be understood that the electronic device 1000 illustrated in FIG. 10 is merely exemplary and should not constitute any limitation on the functionality and scope of the embodiments described herein. The electronic device 1000 shown in FIG. 10 may be configured to implement the server 130, the terminal device 110, the terminal device 160 in FIG. 1, or the apparatus 900 in FIG. 9.

As shown in FIG. 10, the electronic device 1000 is in the form of a general-purpose computing device. Components of the electronic device 1000 may include, but are not limited to, one or more processors or processing units 1010, a memory 1020, a storage device 1030, one or more communication units 1040, one or more input devices 1050, and one or more output devices 1060. The processing unit 1010 may be an actual or virtual processor and capable of performing various processes according to programs stored in the memory 1020. In a multiprocessor system, multiple processing units execute computer-executable instructions in parallel to improve parallel processing capabilities of electronic device 1000.

The electronic device 1000 typically includes a plurality of computer storage media. Such media may be any available media accessible to the electronic device 1000, including, but not limited to, volatile and non-volatile media, removable and non-removable media. The memory 1020 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 1030 may be a removable or non-removable medium and may include a machine-readable medium, such as a flash drive, magnetic disk, or any other medium, which may be capable of storing information and/or data and may be accessed within electronic device 1000.

The electronic device 1000 may further include additional removable/non-removable, volatile/non-volatile storage media. Although not shown in FIG. 10, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 1020 may include a computer program product 1025 having one or more program modules configured to perform various methods or actions of various embodiments of the present disclosure.

The communication unit 1040 is configured to communicate with another electronic device through a communication medium. Additionally, the functionality of components of the electronic device 1000 may be implemented in a single computing cluster or multiple computing machines capable of communicating over a communication connection. Thus, the electronic device 1000 may operate in a networked environment using logical connections with one or more other servers, network personal computers (PCs), or another network Node.

The input device 1050 may be one or more input devices such as a mouse, a keyboard, a trackball, or the like. The output device 1060 may be one or more output devices, such as a display, a speaker, a printer, or the like. The electronic device 1000 may also communicate with one or more external devices (not shown) through the communication unit 1040 as needed, external devices such as storage devices, display devices, etc., communicate with one or more devices that enable a user to interact with the electronic device 1000, or communicate with any device (e.g., a network card, a modem, etc.) that enables the electronic device 1000 to communicate with one or more other electronic devices. Such communication may be performed via an input/output (I/O) interface (not shown).

According to example embodiments of the present disclosure, there is provided a computer-readable storage medium having computer-executable instructions stored thereon, wherein the computer-executable instructions are executed by a processor to implement the method described above. According to an exemplary embodiment of the present disclosure, a computer program product is further provided, the computer program product being tangibly stored on a non-transient computer readable medium and comprising computer executable instructions, the computer executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/acts specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/acts specified in the flowchart and/or block diagram(s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/actions specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are exemplary, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, the practical application, or improvements to the technology in the marketplace, or to enable others of ordinary skill in the art to understand the various embodiments disclosed herein.

I/We claim:

1. A method of information interaction, comprising:
determining at least one question provided by a digital assistant to a user based on an information collection requirement of an electronic medical record;
generating an electronic medical record of a consultation object based at least on interaction information between the user and the digital assistant, at least part of information in the electronic medical record being collected from the interaction information; and
in accordance with a determination that a completeness of the electronic medical record satisfies a predetermined completeness condition, presenting the electronic medical record to medical personnel to determine a subsequent operation for the electronic medical record.

2. The method of claim 1, wherein the electronic medical record at least comprises medical condition information related to the consultation object, and wherein presenting the electronic medical record to medical personnel comprises:
in accordance with a determination that completeness of the medical condition information in the electronic medical record satisfies a predetermined completeness condition, presenting the electronic medical record to medical personnel.

3. The method of claim 1, further comprising:
in response to receiving a confirmation on the electronic medical record by the medical personnel, storing the confirmed electronic medical record in association with the consultation object or the user.

4. The method of claim 3, further comprising:
providing the confirmed electronic medical record in an interaction interface between the user and the digital assistant for viewing by the user.

5. The method of claim 1, further comprising:
in response to receiving a return of the electronic medical record by the medical personnel, initiating a further interaction between the digital assistant and the user; and
generating a modified electronic medical record based on information of the further interaction between the user and the digital assistant.

6. The method of claim 1, further comprising:
in response to receiving an editing operation on the electronic medical record by the medical personnel, providing a medical record editing interface to receive editing content for the electronic medical record.

7. The method of claim 6, wherein the medical record editing interface comprises a viewing entrance for a plurality of interaction messages between the user and the digital assistant, the method further comprising:
presenting the plurality of interaction messages based on a triggering operation on the viewing entrance, the plurality of interaction messages being selectable;
receiving a selection on at least one interaction message in the plurality of interaction messages by the medical personnel;
updating the electronic medical record with the selected at least one interaction message.

8. The method of claim 1, wherein in the electronic medical record, at least one entity word is annotated with a respective entry link, and each entry link is used for accessing an entry page corresponding to an entity word.

9. The method of claim 1, wherein at least one of the following: an access link of the electronic medical record, or an access link of the interaction information is provided in a page associated with the electronic medical record.

10. The method of claim 1, wherein the digital assistant generates a reply for the user based on a model.

11. An electronic device, comprising:
at least one processing unit; and
at least one memory coupled to the at least one processing unit and storing instructions for execution by the at least one processing unit, the instructions, when executed by the at least one processing unit, causing the device to perform acts comprising:
determining at least one question provided by a digital assistant to a user based on an information collection requirement of an electronic medical record;
generating an electronic medical record of a consultation object based at least on interaction information between the user and the digital assistant, at least part of information in the electronic medical record being collected from the interaction information; and
in accordance with a determination that a completeness of the electronic medical record satisfies a predetermined completeness condition, presenting the electronic medical record to medical personnel to determine a subsequent operation for the electronic medical record.

12. The electronic device of claim 11, wherein the electronic medical record at least comprises medical condition information related to the consultation object, and wherein presenting the electronic medical record to medical personnel comprises:
in accordance with a determination that completeness of the medical condition information in the electronic medical record satisfies a predetermined completeness condition, presenting the electronic medical record to medical personnel.

13. The electronic device of claim 11, wherein the acts further comprise:
in response to receiving a confirmation on the electronic medical record by the medical personnel, storing the confirmed electronic medical record in association with the consultation object or the user.

14. The electronic device of claim 13, wherein the acts further comprise:
providing the confirmed electronic medical record in an interaction interface between the user and the digital assistant for viewing by the user.

15. The electronic device of claim 11, wherein the acts further comprise:
in response to receiving a return of the electronic medical record by the medical personnel, initiating a further interaction between the digital assistant and the user; and
generating a modified electronic medical record based on information of the further interaction between the user and the digital assistant.

16. The electronic device of claim 11, wherein the acts further comprise:
in response to receiving an editing operation on the electronic medical record by the medical personnel, providing a medical record editing interface to receive editing content for the electronic medical record.

17. The electronic device of claim 16, wherein the medical record editing interface comprises a viewing entrance for a plurality of interaction messages between the user and the digital assistant, the acts further comprising:
presenting the plurality of interaction messages based on a triggering operation on the viewing entrance, the plurality of interaction messages being selectable;
receiving a selection on at least one interaction message in the plurality of interaction messages by the medical personnel;
updating the electronic medical record with the selected at least one interaction message.

18. The electronic device of claim 11, wherein in the electronic medical record, at least one entity word is annotated with a respective entry link, and each entry link is used for accessing an entry page corresponding to an entity word.

19. The electronic device of claim 11, wherein at least one of the following: an access link of the electronic medical record, or an access link of the interaction information is provided in a page associated with the electronic medical record.

20. A non-transitory computer-readable storage medium having a computer program stored thereon, the computer program being executable by a processor to implement a method for information interaction comprising:
determining at least one question provided by a digital assistant to a user based on an information collection requirement of an electronic medical record;
generating an electronic medical record of a consultation object based at least on interaction information between the user and the digital assistant, at least part of information in the electronic medical record being collected from the interaction information; and
in accordance with a determination that a completeness of the electronic medical record satisfies a predetermined completeness condition, presenting the electronic medical record to medical personnel to determine a subsequent operation for the electronic medical record.
